(19) United States
(12) Patent Application Publication
(10) Pub. No.: US 2017/0035387 A1
(43) Pub. Date: Feb. 9, 2017

(54) CONTACT DETERMINATION APPARATUS

(71) Applicant: KONINKLIJKE PHILIPS N.V., EINDHOVEN (NL)

(72) Inventors: Fei ZUO, EINDHOVEN (NL); Godefridus Antonius HARKS, RIJEN (NL); Caifeng SHAN, VELDHOVEN (NL); Szabolcs DELADI, VELDHOVEN (NL); Harm Jan Willem BELT, WEERT (NL); Jaap KNOESTER, UTRECHT (NL)

(73) Assignee: KONINKLIJKE PHILIPS N.V., Eindhoven (NL)

(21) Appl. No.: 15/304,083

(22) PCT Filed: May 2, 2015
(86) PCT No.: PCT/EP2015/059634

§ 371 Date: Oct. 14, 2016

(30) Foreign Application Priority Data
May 2, 2014 (EP) 14166823.6

(51) Int. Cl. A61B 8/12; A61B 8/08; A61B 18/14; A61B 8/00

(52) U.S. Cl. CPC A61B 8/12; A61B 8/429; A61B 8/5223; A61B 18/1492; A61B 6/487

(57) ABSTRACT

The invention relates to a contact determination apparatus for determining a degree of contact between a device (2) and a moving object (3), wherein the contact determination apparatus comprises a motion distribution providing unit for providing a motion distribution being indicative of motion in the surrounding of the device and a contact determination unit (14) for determining a degree of contact between the device and the object based on the provided motion distribution. Since the degree of contact between the device and the object is determined based on the provided motion distribution and not simply based on, for instance, a peak of an A-mode signal, the determination of the degree of contact can be less disturbed by artifacts like ring-down artifacts or by blood scattering, if the object is, for instance, tissue. This can lead to a more accurate determination of the degree of contact.

### FIELD OF THE INVENTION

The invention relates to a contact determination apparatus, method and computer program for determining a degree of contact between a device like a catheter and a moving object like tissue. The invention relates further to an energy application system like an ablation system for applying energy to the object.

### BACKGROUND OF THE INVENTION

U.S. Pat. No. 5,840,030A discloses an ablation catheter having an ablation electrode for ablating cardiac tissue and a marking ultrasound transducer at a location adjacent to the ablation electrode. A scanner transducer in another device, which may be a transoesophageal echography probe or a transthoracic echocardiographic imaging transducer is adapted to generate A-mode ultrasound signals and the ultrasonic field is directed towards the ablation catheter such that the marking transducer in the ablation catheter is within the ultrasonic field of the scanner transducer. The A-mode signal is used to determine whether the ablation electrode is in intimate contact with the cardiac tissue based on a position of a peak of the A-mode ultrasound signal representing the marking transducer with respect to a position of another peak of the A-mode signal representing the cardiac tissue. Furthermore, it is mentioned that the localization of the position of the ablation electrode with respect to the myocardium may be possible with a similar methodology using Doppler velocity measurement.

Determining whether the ablation electrode is in intimate contact with the cardiac tissue based on the peak of the A-mode signal has the disadvantage that this determination can be disturbed by artifacts like ring-down artifacts or by blood scattering.

### SUMMARY OF THE INVENTION

It is an object of the present invention to provide a contact determination apparatus, method and computer program for determining a degree of contact between a device and a moving object, which allow for a more accurate determination. It is a further object of the present invention to provide an energy application system for applying energy to an object, which comprises the contact determination apparatus.

In a first aspect of the present invention a contact determination apparatus for determining a degree of contact between a device and a moving object is presented, which is operatively coupled to a motion distribution determination unit (13) providing signals indicative of motion in a surrounding of the device (2)

wherein the contact determination apparatus is adapted to determine a variance of the velocity in the surrounding of the device (2) comprising the moving object (3) from the signals received from the motion distribution determination unit (13), and

wherein the contact determination apparatus is configured to determine a degree of contact between the device and the object based on the variance of the velocity.

Since the degree of contact between the device and the object is determined based on the variance of the velocity and not simply based on a peak of an A-mode signal, the determination of the degree of contact can be less disturbed by artifacts like ring-down artifacts or by blood scattering. This can lead to a more accurate determination of the degree of contact.

The object is preferentially tissue, especially cardiac tissue. It may move due to cardiac contraction, catheter motion and/or tissue compression by the device. The device is preferentially a catheter. The motion distribution determination unit is preferentially adapted to provide a motion distribution covering a distance of several tens of millimeters from the device. The motion distribution preferentially describes the motion of elements in the surrounding of the device. For instance, the velocities of blood and/or tissue in the surrounding of the device may be provided as the motion distribution. Moreover, the motion distribution determination unit is preferentially adapted to provide the motion distribution over time, wherein the contact determination apparatus is preferentially adapted to determine a degree of contact between the device and the tissue over time based on variance of the velocity.

The motion distribution determination unit may be a storing unit, in which the motion distribution is stored and from which the stored motion distribution can be provided to the contact determination apparatus. However, the motion distribution determination unit can also be a receiving unit for receiving the motion distribution from another unit having determined the motion distribution and for providing the received motion distribution to the contact determination apparatus. Moreover, the motion distribution determination unit can be adapted to determine the motion distribution based on ultrasound signals received from an ultrasound transducer based on, for instance, the Doppler effect and to provide the determined motion distribution. The ultrasound transducer can also be regarded as being a part of the motion distribution determination unit such that the motion distribution determination unit generates the ultrasound signals and determines the motion distribution based on the generated ultrasound signal. One or more ultrasound transducers may be used for determining ultrasound signals in one or several directions, respectively, wherein in each direction a motion distribution may be determined and for each direction a degree of contact may be determined. In an embodiment four ultrasound transducers are used for generating ultrasound signals in four different directions, wherein the motion distribution is determined in these four directions and for each of these directions the degree of contact is determined. The one or several ultrasound transducers are preferentially integrated in the device.

The motion distribution is preferentially a distribution of motion values, wherein the motion values are related to the motion in the surrounding of the device. The motion values are preferentially velocity values or values which depend on the velocity values, especially which are derivable from the velocity values. In particular, the motion distribution providing unit is preferentially adapted to provide a distribution of the velocity and/or of the mean velocity, wherein the velocity preferentially relates to the velocity of elements like tissue and/or blood in the surrounding of the device.

The contact determination apparatus is adapted to determine a variance of the velocity in a surrounding of the device comprising the moving object from the signals received from the motion distribution determination unit. The variance of the velocity confers several advantages with respect to only using the Doppler velocity for determining a degree of contact between the device and the moving object such as cardiac tissue. The velocity of the cardiac tissue due to repetitive cardiac contraction may have similar values as the velocity of the blood surrounding the device and located between the cardiac tissue and the device. In this case there cannot be distinguished between the device contacting blood or cardiac tissue based on the magnitude of the velocities.

The variance of the velocity measures how far a set of velocity values is spread out. A small variance of the velocity indicates that the velocity values tend to be very close to the mean velocity and hence to each other, while a large variance of the velocity indicates that the velocity values are broadly spread out around the mean and from each other. Blood and cardiac tissue have different physical properties such as density and state of matter, as a consequence the variance of the velocity values in blood and cardiac tissue present different and distinguishable characteristics in a surrounding of the device, wherein the blood and cardiac tissue are in motion due to the repetitive cardiac contractions, even if velocity values of blood and cardiac tissue comprise values of similar magnitude.

The contact determination apparatus may be adapted to determine a fraction defined by the ratio between the number of variance of the velocity values being larger than a first threshold and the total number of variance of the velocity values in a depth-time window, wherein the degree of contact is based on the determined fraction. In an embodiment the fraction is directly used as the degree of contact. When the fraction with values between 0 and 1 has a large value, then the number of variance of the velocity values larger than the first threshold is high and therefore the likelyhood of no contact between the device and the cardiac tissue is high. Moreover, in an embodiment the contact determination apparatus is adapted to determine the degree of contact by thresholding the determined fraction. For instance, in an embodiment the contact determination unit is adapted to provide a second threshold and a third threshold being larger than the second threshold, to determine that the degree of contact is a) a continuous contact, if the fraction is smaller than the second threshold, b) no contact, if the fraction is larger than the third threshold, and c) an intermittent contact otherwise. The first threshold is a variance of the velocity value which can be set dependent on the circumstances of the motion in the surrounding of the device, whereas the second and the third thresholds have values between 0 and 1, and are related to the fraction. The first, second and third thresholds may be determined by calibration measurements. The contact determination apparatus is adapted to determine the degree of contact by using only a part of the motion distribution. For instance, if the motion distribution is a spatial and temporal distribution, then a depth-time window can be defined and only variance of the velocity values of the motion distribution within the window can be used for determining the degree of contact. The fraction of the number of variance of the velocity values larger than the first threshold from the total number of variance of the velocity values may be determined for the depth-time window. The window can be predefined and/or it can be temporally moved, in order to determine several degrees of contact for different temporal positions, i.e. for different times, such that the degree of contact can be monitored over time.

In an embodiment the device may have integrated ultrasound transducers operatively coupled to the motion determination unit and which are capable of generating ultrasound signals in four different directions, wherein in each direction a motion distribution is determined and the contact determination apparatus is configured to determine for each direction a degree of contact between the respective ultrasound transducer and the object based on the respective variance of the velocities. In this case the contact determination apparatus may be adapted to determine that the degree of contact is a) a continuous or stable contact, if for at least one of the ultrasound transducers a continuous or stable contact has been determined, b) an intermittent contact, if for none of the ultrasound transducers a continuous or stable contact and for at least one of the ultrasound transducers an intermittent contact has been determined, and c) no contact, if for none of the ultrasound transducers a continuous or stable or intermittent contact has been determined. This allows for a reliable determination of the degree of contact between the energy application element and the object. The energy application element may be adapted to apply electrical energy and/or ultrasound energy and/or optical energy to the object. Preferentially, the energy application element is adapted to provide ablation energy to tissue, in order to ablate the tissue. In a preferred embodiment the energy application element is an ablation electrode.

In further aspect of the present invention an energy application system for applying energy to an object is presented, wherein the energy application system comprises: an energy source, an energy application device for applying energy to the object, a motion distribution determination unit, and the contact determination apparatus,

wherein the energy application device is operatively coupled to the energy source,

wherein the energy application device further comprises an ultrasound transducer operatively coupled to the motion distribution determination unit,

wherein the contact determination apparatus is configured to determine the degree of contact between the energy application device and the object based on the variance of the velocity.

In an embodiment of the energy application system the energy source is adapted to provide energy to the energy application device only when the contact determination apparatus determines a continuous contact between the energy application device and the object. In a further embodiment the energy application system may also comprise a navigation unit.

In a further aspect of the present invention a contact determination method for determining a degree of contact between a device and a moving object is presented, wherein the contact determination method comprises:

providing motion distribution signals by a motion distribution determination unit, the signals being indicative of the motion in the surrounding of the device comprising an object,

determining a variance of the velocity from the motion distribution signals, and

determining a degree of contact between the device and the object based on the variance of the velocity.

An energy application method may comprise the following steps:

navigating the energy application device to the object,

determining the degree of contact between the energy application device and the object based on the variance of the velocity, and

performing energy application to the object when a continuous contact between the energy application device and the object is determined.

In a further aspect of the present invention a computer-readable medium having stored a computer-executable program for determining a degree of contact between a device and a moving object is presented, wherein the computer program comprises program code means for causing a contact determination apparatus to carry out the steps of the contact determination method, when the computer program is run on a computer controlling the contact determination apparatus.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an energy application system for applying energy to an object,

FIG. 2 shows schematically and exemplarily an embodiment of a tip of an ablation catheter of the energy application apparatus shown in FIG. 1,

FIG. 3 shows an M-mode ultrasound image and the corresponding variance of the velocity distribution rendered in depth-time coordinates,

FIG. 4 shows schematically and exemplarily a depth-time window moving in time on the rendered variance of the velocity distribution,

FIG. 5 shows schematically and exemplarily an embodiment of a contact determination method for determining a degree of contact between a device and a moving object,

FIG. 6 schematically and exemplarily illustrates a Doppler-based motion determination procedure,

FIG. 7 shows schematically and exemplarily an embodiment of an energy application method for applying energy to an object,

FIGS. 8 to 11 schematically and exemplarily show M-mode images and variance of the velocity distributions, and

FIG. 12 shows a flowchart exemplarily illustrating an embodiment of processing A-mode lines for determining a degree of contact between a device and an object.

### DETAILED DESCRIPTION OF EMBODIMENTS

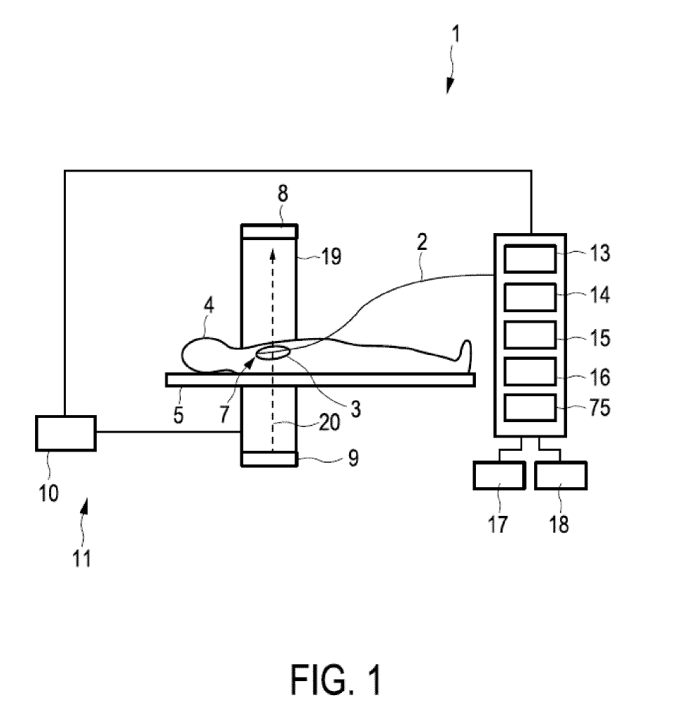

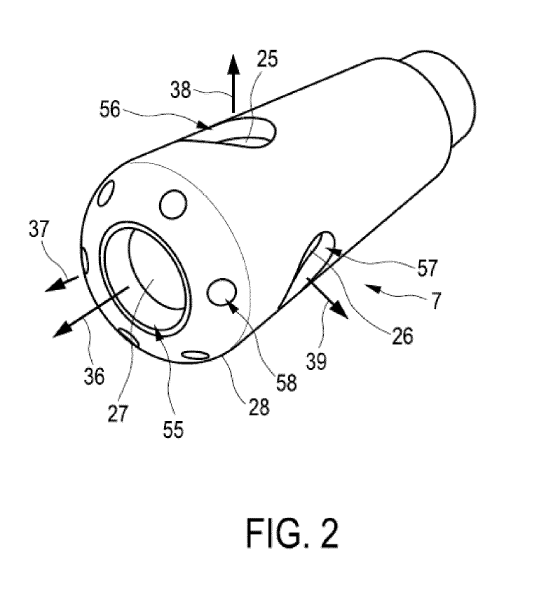

FIG. 1 shows schematically and exemplarily an embodiment of an energy application system for applying energy to an object. In this embodiment the energy application system 1 is an ablation system for ablating cardiac tissue within a heart 3 of a person 4 lying on a support means 5 like a patient table. An energy application device 2 being an ablation catheter has been introduced into the person 4 such that the tip 7 of the ablation catheter is arranged within the heart 3. FIG. 2 shows schematically and exemplarily the tip 7 of the ablation catheter 2 in more detail.

The tip 7 of the ablation catheter 2 comprises four ultrasound transducers and an ablation electrode 28, wherein from the fours ultrasound transducers only three ultrasound transducers 25, 26, 27 are visible in FIG. 2. The four ultrasound transducers are electrically connected with a motion distribution determination unit 13, which is adapted to control the four ultrasound transducers and to determine four motion distributions for four different directions 36 . . . 39, for which the four ultrasound transducers generate ultrasound signals. In this embodiment the four directions include an axial direction 36 and three lateral directions 37, 38, 39. The ablation electrode 28 comprises an axial opening 55 and three lateral openings, wherein from these three lateral openings only two lateral openings 56, 57 are visible in FIG. 2. The axial and lateral openings allow the ultrasound transducers to send and receive ultrasound waves along the four different directions 36 . . . 39. Moreover, the ablation electrode 28 comprises irrigation openings 58 for allowing irrigation fluid, which may flow within the ablation catheter 2, to leave the tip 7 of the ablation catheter 2.

The determination of the four motion distributions is preferentially performed by using known Doppler-based motion determination techniques. The four ultrasound signals are preferentially A-mode signals generated over time such that for each direction a motion distribution may be determined over time. Moreover, for each direction an M-mode image can be provided. Since the four ultrasound transducers and the motion distribution determination unit 13 are used for determining motion distributions, which can be provided to a contact determination apparatus 14, the four ultrasound transducers and the motion distribution determination unit 13 can be regarded as being a motion distribution providing unit for providing motion distributions, i.e. in the present embodiment four motion distributions, being indicative of the motion in the surrounding of the tip 7 of the ablation catheter 2.

The motion distribution providing unit can be adapted to provide, for instance, a distribution of the velocity and/or of the mean velocity, wherein the velocity preferentially relates to the velocity of elements like tissue and/or blood in the surrounding of the device. In particular, the contact determination apparatus can be adapted to determine the velocity spectral variance, i.e. the variance of the velocity, in accordance with following equation:

$$fb = \frac{\sum {( f - fc )}^{2} P ( f )}{\sum P ( f )} , \qquad ( 1 )$$

wherein f is a frequency, fc is a center frequency and P(f) is the velocity spectrum. For more details regarding this determination of the variance of the velocity reference is made to the article “Real-Time Two-Dimensional Blood Flow Imaging Using an Autocorrelation Technique” by C. Kasai et al., IEEE Transactions on Sonics and Ultrasonics, volume 32, number 3, pages 458 to 464 (1985), which is herewith incorporated by reference.

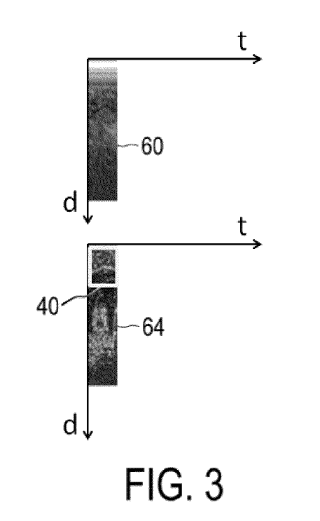

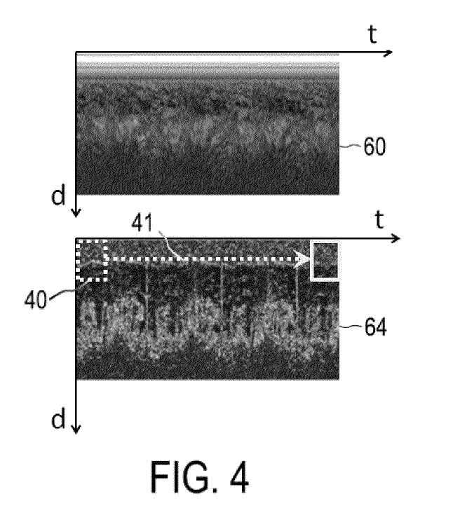

The contact determination apparatus is adapted to determine the velocity spectral variances, i.e. variances of the velocity, in order to provide a measure of the spread of the spectrum for each location at a given time point, i.e. for each pixel in the two-dimensional depth-time plot. The velocity spectral variance is preferentially determined in accordance with equation (1). An alternative is to calculate the variance or standard deviation of the mean spectral frequency, i.e. of the mean velocity, within a region of interest. Based on this, a plot of the variance of the velocities 64 can be rendered as presented in FIG. 3, where each pixel indicates a variance of the velocity that has a one-to-one correspondence to the ultrasound information of the original M-mode image 60. The region of interest may be a depth-time window 40 extending from the surface of the transducer into the surroundings in the spatial direction and comprising a time interval in the temporal direction, preferable at least the duration between two consecutive heart contractions. The depth-time window 40 may be moved 41 in time as shown in FIG. 4, i.e. it may be a moving window, in order to determine the variance of the velocity for different positions in time. Alternatively the depth-time window may be defined on the M-mode ultrasound image 60 or through the input unit 17 by indicating the spatial and temporal dimensions for which the contact determination apparatus should determine the variance of the velocity.

In an embodiment the depth-time window comprises a number of variance of velocity values, i.e. for each pixel in the depth-time window. The contact determination apparatus 14 is preferentially further adapted to determine the fraction of the number of the variance of the velocity values being larger than a first threshold from the total number of the variance of the velocity values and to determine the degree of contact in the respective direction based on the determined fraction. In particular, the contact determination apparatus 14 is adapted to provide a second threshold and a third threshold, which may be larger than the second threshold, and to determine that the degree of contact is a) a continuous or stable contact, if the fraction is smaller than the second threshold, b) no contact, if the fraction is larger than the third threshold, and c) an intermittent contact otherwise. The intermittent contact preferentially describes a situation in which there is alternating contact and no contact, especially for a given time period during which the tissue should be ablated. The first threshold is a variance of the velocity value which can be set dependent on the circumstances of the motion in the surrounding of the device, whereas the second and the third thresholds have values between 0 and 1, and are related to the fraction. The first, second and third thresholds may be determined by calibration measurements, wherein the motion distributions are determined while the degree of contact is known, and wherein during the calibration the first, second and third thresholds are adjusted such that the known degree of contact is reliably determined by the contact determination apparatus 14.

The contact determination apparatus 14 is preferentially further adapted to determine a degree of contact between the ablation electrode and the cardiac tissue based on the determined degrees of contacts between the ultrasound transducers and the cardiac tissue. In particular, the contact determination apparatus 14 may be adapted to determine that the ablation electrode is in a stable or continuous contact with the cardiac tissue, if for at least one of the four ultrasound transducers a continuous or stable contact has been determined, to determine an intermittent contact between the ablation electrode and the cardiac tissue, if for none of the four ultrasound transducers a continuous or stable contact and for at least one of the four ultrasound transducers an intermittent contact has been determined, and to determine that there is no contact between the ablation electrode and the cardiac tissue, if for none of the ultrasound transducers a stable or continuous or intermittent contact has been determined.

The ablation system 1 further comprises an RF energy source 15, which is electrically connected with the ablation electrode at the tip 7 of the ablation catheter 2 and which is adapted to provide RF energy to the ablation electrode, in order to allow the ablation electrode to ablate the cardiac tissue. Moreover, the ablation system 1 may further comprise a navigation unit 16 for navigating the catheter 2 within the person 4 by using, for instance, known navigation techniques which may be based on steering wires and/or robotic means. The ablation apparatus 1 may further comprise an irrigation control unit 75 for controlling the flow of the irrigation fluid within the ablation catheter 2 and, thus, for controlling the irrigation fluid leaving the tip 7 of the ablation catheter 2 through the irrigation openings 58. The ablation catheter 2 comprises a lumen for guiding the fluid from the irrigation control unit 75 to the irrigation openings 58. The irrigation control unit 75 preferentially comprises a fluid source and a pump for providing fluid to the tip 7 of the ablation catheter 2.

The ablation system 1 further comprises a fluoroscopy device 11 for imaging the tip 7 of the ablation catheter 2 within the person 4 during the interventional procedure. The fluoroscopy device 11 comprises an x-ray source 9 for emitting x-rays 20 traversing the person 4 lying on the support means 5. The fluoroscopy device 11 further comprises an x-ray detector 8 for detecting the x-rays 20, after having traversed the person 4. The x-ray source 9 and the x-ray detector 8 are mounted on a C-arm 19, which is rotatable with respect to the person 4, in order to irradiate the person 4 in different directions. Moreover, the support means 5 and the C-arm 19 may be translatable with respect to each other, in order to irradiate different parts of the person 4. The x-ray detector 8 is adapted to generate detection signals being indicative of the detected x-rays 20, wherein the detection signals are transmitted to a fluoroscopy control unit 10, which is adapted to control the C-arm 19, the x-ray source 9 and the x-ray detector 8 and to generate two-dimensional projection images depending on the received detection signals. The two-dimensional projection images may be shown on a display 18, in order to allow a user like a physician to monitor the position of the tip 7 of the ablation catheter 2 within the person 4 during the interventional procedure. In other embodiments other tracking/navigating techniques can be used for monitoring the position of the tip 7 of the ablation catheter 2 during the interventional procedure like optical shape sensing based tracking techniques or electromagnetic tracking techniques.

The ablation system further comprises an input unit 17 for allowing the user to input commands like navigational commands, energy application commands, et cetera. For instance, the determined degree of contact between the ablation electrode and the cardiac tissue may be shown on the display 18, whereupon a user may input a command indicating that the ablation energy should be applied to the cardiac tissue by using the input unit 17, if the determined degree of contact indicates a stable or continuous contact between the ablation electrode and the cardiac tissue. In an embodiment the RF energy source 15 may be adapted to provide RF energy only, if the contact determination unit 14 has determined that the degree of contact is a stable or continuous contact.

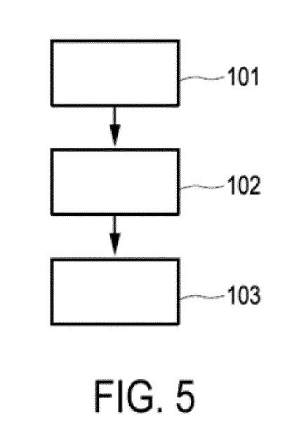

In the following an embodiment of a contact determination method will exemplarily be described with reference to a flowchart shown in FIG. 5.

In step 101 the motion distribution determining unit provides a motion distribution in the surrounding of the tip of the ablation catheter. In particular, four ultrasound transducers are used for generating A-mode signals for four different directions over time, wherein these A-mode signals are used for determining for the four different directions four motion distributions, i.e. velocity distributions.

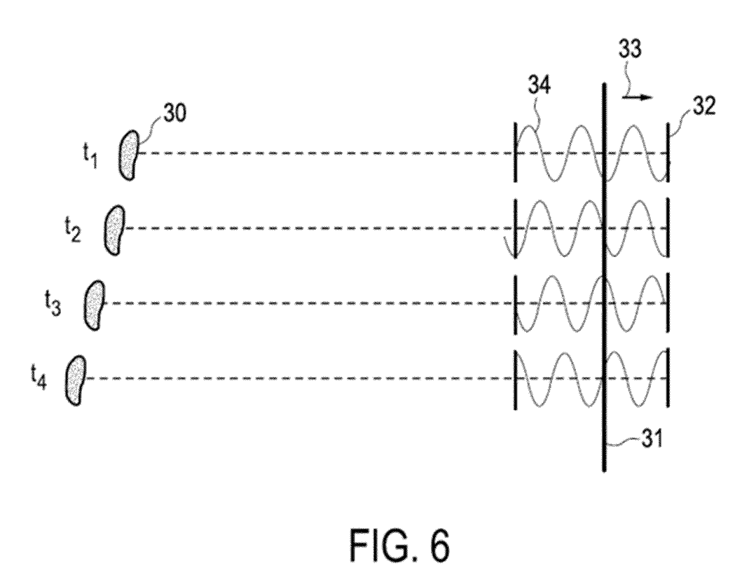

One way to determine a velocity distribution is a determination of the velocity of scatters from the ultrasound signals by using known Doppler techniques such as used in color-flow imaging. The Doppler techniques can be adapted to determine the phase shift rate between A-mode lines, for instance between corresponding ultrasound RF lines, and to determine the respective velocity based on the determined phase shift rate. The change of the phase shift is exemplarily illustrated in FIG. 6, wherein ultrasound waves 34 scattered by the element 30 propagate in the direction indicated by the arrow 33 and are received by the ultrasound transducer 32. At different times t₁ . . . t₄ the element 30 is located at different positions due to the motion of the element 30, wherein the different positions of the element 30 at the different times t₁ . . . t₄ lead to phase shifts in the ultrasound waves 34, for instance, at the line 31. Based on these phase shifts the velocity of the element 30 can be determined by using, for instance, the Doppler technique disclosed in the article “Ultrasonic colour Doppler imaging” by D. H. Evans et al., Interface Focus 1(4), pages 490 to 502 (2011), which is herewith incorporated by reference.

In step 102 the contact determination apparatus determines the variance of the velocity values from the provided motion distributions and determines a degree of contact between the ablation catheter, especially the ablation electrode and the cardiac tissue based on the variance of the velocity. Preferentially, for each ultrasound transducer a degree of contact is determined and the degree of contact between the tip of the ablation catheter and the cardiac tissue is determined based on the degrees of contact determined for the ultrasound transducers. In step 103 the determined degree of contact is shown on the display.

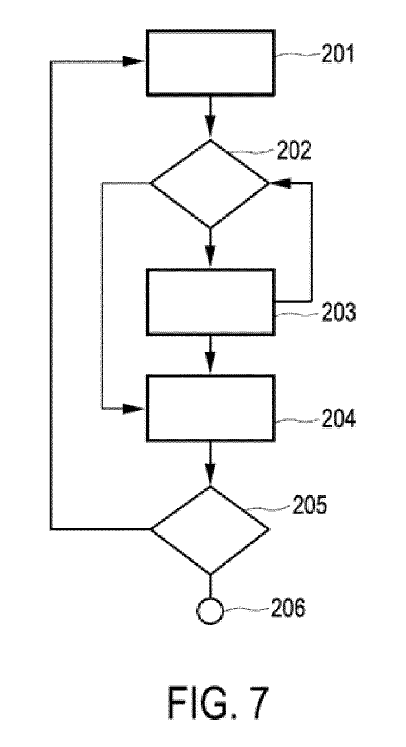

In the following an embodiment of an energy application method for applying energy to an object will exemplarily be described with reference to a flowchart shown in FIG. 7. In this embodiment the energy application method is an ablation method for ablating cardiac tissue by using an ablation electrode at a tip of an ablation catheter.

In step 201 the tip of the ablation catheter is navigated to the cardiac tissue to be ablated under guidance of the fluoroscopy device. In step 202 it is determined whether there is a desired degree of contact between the ablation electrode and the cardiac tissue. In particular, it is determined whether there is a stable or continuous contact between the ablation electrode and the cardiac tissue. The degree of contact is determined as described above with reference to, for instance, FIG. 5. If there is no desired degree of contact between the ablation electrode and the cardiac tissue, in step 203 the position of the tip of the ablation catheter within the cardiac tissue may be modified, where after it can again be checked whether there is a desired degree of contact between the ablation electrode and the cardiac tissue. If there is a desired degree of contact between the ablation electrode and the cardiac tissue, in step 204 the cardiac tissue can be ablated. In step 205 a user may indicate whether the tip of the ablation catheter should be moved to another ablation site at which the cardiac tissue should be ablated, wherein, if this is the case, the method continues with step 201, in which the tip of the ablation catheter is moved to the other ablation site. If the tip of the ablation catheter does not need to be moved to a further ablation site, the method ends in step 206.

The ultrasound transducers integrated in the tip of the ablation catheter provide ultrasound signals for monitoring the ablation procedure in real-time. Prior to the ablation procedure, which is preferentially performed for treating cardiac arrhythmia, the catheter tissue contact can be assessed. A good catheter-tissue contact is an important factor that can influence the final ablation outcome. In an embodiment the motion distribution determination unit and the contact determination apparatus described above with reference to, for instance FIG. 1, may form a system for determining a degree of contact between the tip of the ablation catheter and the cardiac tissue, which may allow for providing an automatic contact indicator purely based on the ultrasound signals, in order to assist the user, especially a physician, when navigating the catheter to the desired site and to guide him/her to ensure a good catheter/tissue contact before performing the ablation procedure.

The contact determination apparatus can be adapted to distinguish cardiac tissue within the field of view of the respective ultrasound transducer from other elements, which may be visible in the respective ultrasound signal like irrigation fluid, noise, blood scatters, ring-down artifacts, et cetera.

A more traditional way to access the tissue presence from ultrasound signals or ultrasound images is to look at the signal strength or amplitude, for instance, as disclosed in U.S. Pat. No. 5,840,030A. However, this kind of assessing the tissue presence has several drawbacks. For instance, horizontal artifacts like ring-down artifacts may extend to a variable depth, which can interfere with the signal detection. Moreover, blood scattering can also give a strong signal in an echo image, which may confuse the detection algorithm. This may be particularly problematic for high-frequency ultrasound transducers, because blood scatters high-frequency ultrasound much more than low-frequency ultrasound. Furthermore, even if the tissue is in contact with the tip of the ablation catheter, the signal may be relatively weak. In addition, time gain compensation (TGC) settings can influence the signal strength or amplitude and makes the detection difficult, and, as a result of the cardiac motion, a large catheter motion does not lead to a stable contact, i.e. a stable or continuous contact between the ablation catheter and the cardiac tissue is not correlated with the signal strength or amplitude. Determining a degree of contact between the ablation catheter and the cardiac tissue only based on the signal strength or amplitude, in particular, only based on the position of a peak of an ultrasound signal, as suggested, for instance, in U.S. Pat. No. 5,840,030A, may therefore yield inaccurate results.

In U.S. Pat. No. 5,840,030A it is mentioned that the localization of the position of the ablation electrode with respect to the myocardial wall may be possible using a similar methodology for Doppler velocity measurement, by looking at the velocity amplitudes. The velocity of the cardiac tissue due to repetitive cardiac contractions may have similar values as the velocity of the blood surrounding the device and located between the cardiac tissue and the device. In this case there cannot be distinguished between the device contacting blood or cardiac tissue based on the magnitude of the velocities.

The contact determination apparatus adapted to determine a variance of the velocity in a surrounding of the device comprising the moving object from the signals received from the motion distribution determination unit and further adapted to determine a degree of contact between the device and the moving object based on the variance of the velocity confers a significant advantage with respect to using the Doppler velocity amplitude for determining contact. The variance of the velocity measures how far a set of velocity values is spread out. A small variance of the velocity indicates that the velocity values tend to be very close to the mean velocity and hence to each other, while a large variance of the velocity indicates that the velocity values are broadly spread out around the mean and from each other. Blood and cardiac tissue have different state of matter, as a consequence the variance of the velocity values in blood and cardiac tissue present different and distinguishable characteristics in a surrounding of the device, wherein the blood and cardiac tissue are in motion due to the repetitive cardiac contractions, even if the velocity values of blood and cardiac tissue comprise values of similar magnitude.

The contact determination apparatus hence uses the variance of the velocities in the surrounding of the ablation catheter, which are less influenced by interferences, noises, blood scattering or losses of signal strength from various situations or not influenced at all by these effects.

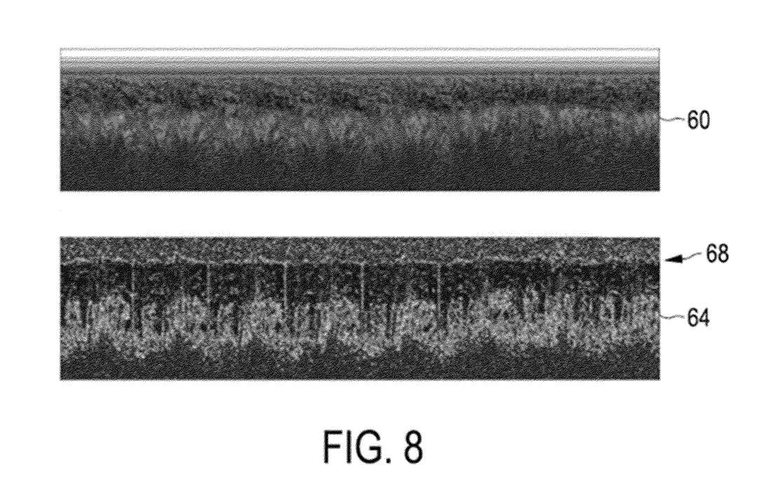

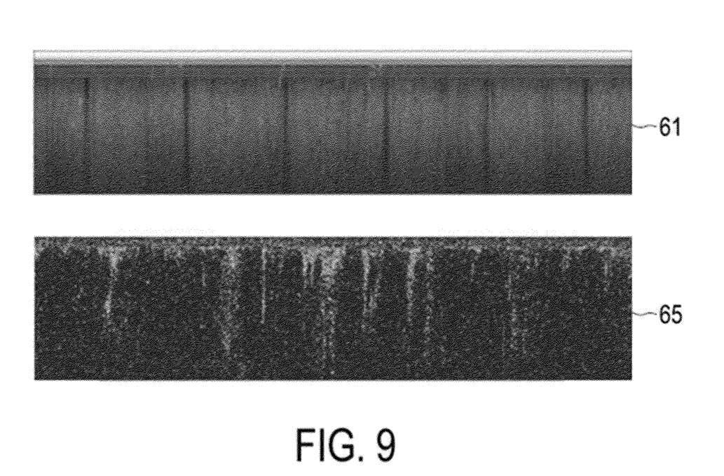

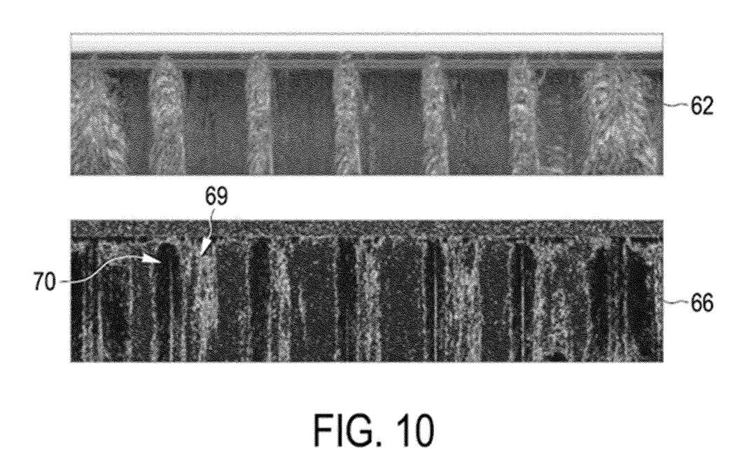

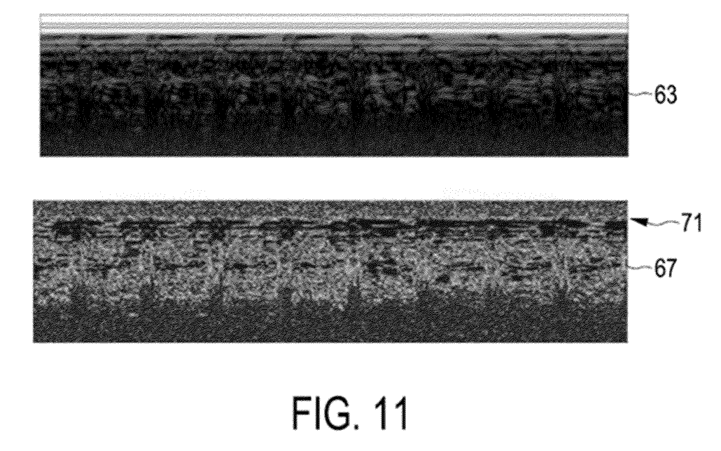

FIGS. 8 to 11 show M-mode images 60 . . . 63 and corresponding variance distributions 64 . . . 67, i.e. distributions of variances of the velocity. In FIG. 8 the surface of the cardiac tissue is located at the position indicated by the arrow 68. As can be seen in FIG. 8, the surface of the cardiac tissue is well recognizable in the variance distribution 64. In FIG. 9 a blood pool has been imaged leading to a relatively homogeneous variance distribution 65. When measuring the M-mode image 62 shown in FIG. 10, the ultrasound transducer was intermittently in contact with the cardiac tissue, wherein blood regions 69 are well distinguishable from tissue regions 70 in the variance distribution 66. When generating the M-mode image 63 shown in FIG. 11, the ultrasound signals were relatively weak. However, the position 71 of the surface of the cardiac tissue is still well recognizable in the variance distribution 67. Since by using the variance distributions the position of the surface of the cardiac tissue relative to the position of the respective ultrasound transducer can clearly be determined, based on the variance distributions it can accurately be determined whether the tip of the ablation catheter is in continuous contact or in intermittent contact or does not have any contact with the cardiac tissue. In particular, blood is well distinguishable from cardiac tissue, because blood shows a more scattered distribution of velocities at the tip of the ablation catheter than cardiac tissue.

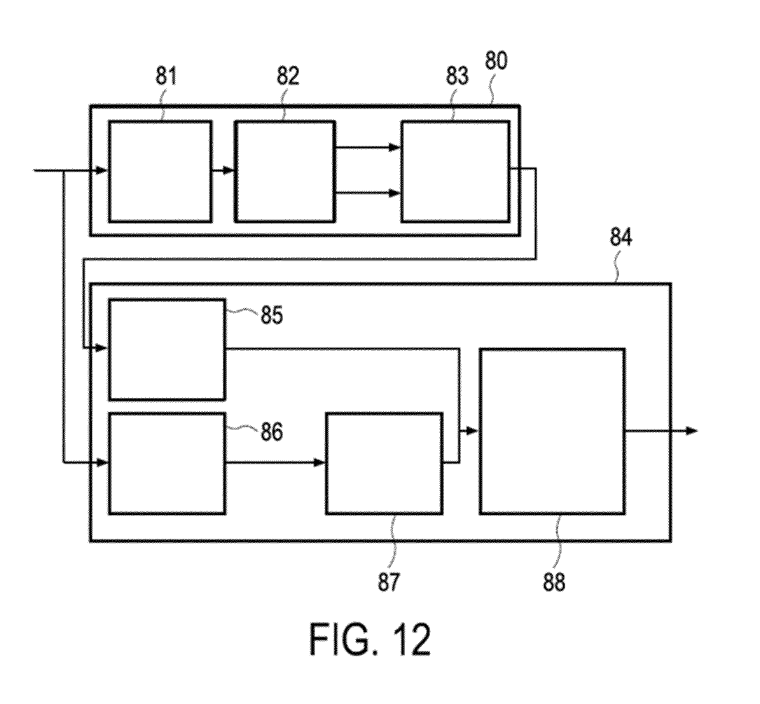

In an embodiment each ultrasound transducer generates A-mode lines, which may also be regarded as being RF lines, wherein these A-mode lines may be processed as will in the following be described with reference to FIG. 12.

In step 80 the A-mode lines are received and a variance distribution, i.e. a distribution of the variance of the velocity, and optionally also a spectral mean distribution, i.e. a distribution of the mean velocity, are determined. In particular, in step 81 a DC removal filter is applied to the A-mode lines, in order to remove any “horizontal” ring-down artifacts in the A-mode lines. This DC removal filter can be regarded as being a ring-down lateral DC removal filter or as being a “horizontal filter”, because it removes the ‘horizontal’ lines from the M-mode image. An embodiment of such a filter will be described in the following, in which s ₗ[n] denotes a sample of the l-th A-mode line data, where l is a line index (l=0,1, . . . , ∞) and n a sample index within one A-mode line (n=0,1, . . . , N-1).

Suppression of the unwanted interface reflection can be achieved on the basis of the observation that they are static across subsequent A-mode lines. For uₙ[l]=s ₗ[n] with l=0,1, . . . , ∞ defined as the A-mode signal progressing over time for one given depth, the unwanted interface reflection is a DC component, which can be suppressed by a DC-removal filter. Such a DC-removal filter may be implemented at every depth of interest, for instance, within the first 2 mm, using following equation:

u′ₙ[l]=α·u′[l−1]+β·uₙ[l]−β·u_{n|l 1|}, (2)

where finally the DC-free A-mode line signal is given by s′ₗ[n]=u′ₙ[l]. The recursion parameter a determines the effective memory of the filter. The parameter a is designed to result in an effective memory of r seconds, and the parameter β is designed to give a 0 dB response at the higher frequencies. Preferentially, they are defined by following equations:

$$\begin{matrix}α = \frac{τ \cdot {f}_{PRF} - 1}{τ \cdot {f}_{PRF}} and & ( 3 ) \\ β = \frac{τ \cdot {f}_{PRF} - 0.5}{τ \cdot {f}_{PRF}} . & ( 4 )\end{matrix}$$

A typical value for τ is τ=1 s, which has proven to be a good value for the removal of the DC components while leaving the desired signal of moving tissue or blood intact.

In step 82 a quadrature detection procedure is applied to the filtered A-mode lines. In particular, an IQ demodulation or Hilbert transform is applied to the filtered A-mode lines. The quadrature detection splits the filtered A-mode lines into two signals, which are used in step 83 for calculating a spectral variance, i.e. the variance of the velocity, and optionally also a spectral mean, i.e. the mean velocity, based on an auto-correlation procedure. Quadrature detection and hence the determination of the quadrature components are well known in the field of digital signal processing. They are disclosed, for instance, in the standard book “Digital signal processing, mathematical and computational methods, software development and applications” by J. M. Blackledge, Horwood, ISBN 1904275265 (2006), especially on pages 131 to 134, which is herewith incorporated by reference.

For determining the mean velocity, M samples over time may be used, i.e. M temporally adjacent A-mode lines, wherein the lateral sampling frequency may be 2 kHz. The mean velocity, i.e. the spectral mean, may then be determined in accordance with following equation:

$$\overline{v} = \frac{{c}_{0}}{2 {t}_{PRF} {ω}_{c} cos θ} . \\ {tan}^{- 1} ( {\sum }_{m = 2}^{M} Q ( m ) I ( m - 1 ) - Q ( m - 1 ) I ( m ) / {\sum }_{m = 1}^{M - 1} I ( m ) I ( m - 1 ) + Q ( m ) Q ( m - 1 ) ) \qquad ( 5 )$$

In equation (5) I and Q are the quadrature components derived from the A-mode lines and M is the packet size, i.e. the number of temporally adjacent A-mode lines used for calculating a mean velocity. The packet size M is, for instance, eight, which means that the phase shift may be investigated over eight consecutive A-mode lines, in order to determine the mean velocity for a certain time being, for instance, the average of the times of the A-mode lines used for determining the average velocity. Moreover, in equation (5) c₀ indicates the speed of sound, t_{PRF} indicates the A-mode line sample period determined by the pulse repetition frequency (PRF), ω_{c} denotes the transducer center frequency and θ denotes the angle between the transducer beam direction and the velocity direction, which is zero in this embodiment. The variance of the velocity may be given by:

$${σ}^{2} = \frac{{c}_{0}}{{t}_{PRI}^{2} {ω}_{c} cos θ} . \\ [ 1 - \frac{1}{w} \{ {[ \frac{1}{M - 1} {\sum }_{m = 1}^{M - 1} Q ( m ) I ( m - 1 ) - Q ( m - 1 ) I ( m ) ]}^{2} + {{[ \frac{1}{M - 1} {\sum }_{m = 1}^{M - 1} I ( m ) I ( m - 1 ) + Q ( m - 1 ) I ( m ) ]}^{2} \}}^{\frac{1}{2}} ] \\ with \\ w = \frac{1}{M - 1} {\sum }_{m = 0}^{M - 1} {Q}^{2} ( m ) + {I}^{2} ( m ) . \qquad ( 6 )$$

For more details regarding the determination of the mean velocity and the variance of the velocity reference is made to the article “Real-time two-dimensional blood flow imaging using an autocorrelation technique” by C. Kasai et al., IEEE Transactions of Sonics and Ultrasonics, volume 32, number 3, pages 458 to 464 (1985), which is herewith incorporated by reference.

In an embodiment step 81 may be omitted, wherein in this case in step 83 the signals generated in step 82 are DC corrected and in step 83 the auto-correlation procedure is performed on the DC-corrected signals.

The spectral variances calculated in step 83 are post-processed in step 85. In particular, in step 83 two-dimensional spectral variance distributions may have been determined, which may still be noisy. The post-processing may be performed such that the noise is reduced in the two-dimensional spectral variance distributions. The post-processing can include a normalization and a median filtering. In step 86 a deaf zone detection procedure is applied to the A-mode lines. In the A-mode lines the deaf zone may vary between, for instance, 0.4 to 0.7 mm. Only beyond this depth the ultrasound transducer can start to pick up ultrasound signals. In order to cope with this variance, the deaf zone detection procedure can be used, in order to determine the exact depth where the ultrasound signal is expected. If in an embodiment because of the known ablation catheter design the deaf zone is fixed, the deaf zone detection procedure can simply provide a constant number like, for instance, 0.4 mm. The A-mode lines processed in step 86 may be decimated signals (100 Hz) which have been analogously TGC corrected in the dB domain.

In an embodiment the deaf zone detection procedure checks every A-line from an M-mode image, i.e. along each A-line it is searched for a significant signal intensity drop. This is based on the assumption that the deaf-zone is usually characterized by a significant higher signal amplitude than the real valid signals. A threshold can be used to detect the “significant signal intensity drop”, wherein this threshold may be predetermined by a calibration procedure which is performed while the location of the deaf zone is known. During the deaf zone detection procedure a low-pass filter may be applied to the A-line, in order to smooth the signal to avoid false detections.

In step 87 a variable check window procedure is performed. In particular, a check window is determined based on the detected deaf zone location. The check window defines the values which should be used for determining the degree of contact between the tip of the ablation catheter and the cardiac tissue. Based on the deaf-zone location the check window can be correspondingly adjusted instead of being fixed in advance. For example, if during performing the deaf zone detection procedure an interface between the deaf zone and a real signal start has been determined as being located at 0.7 mm, the check window can be defined as being located between 0.7 mm to 1.7 mm, if a 1 mm wide check window should be used.

In step 88 a decision logic determines the degree of contact between the tip of the ablation catheter and the cardiac tissue based on the post-processed spectral variance values generated in step 85 and based on the result of step 87. In particular, the decision logic may take a depth-time window, where the temporal dimension of the window is 2 s as input, in order to capture the variance of contact over a full heart contraction cycle. This is especially helpful for judging the intermittent contact, where it could be shown that the tip of the ablation catheter is touching the cardiac tissue only within a specific phase in a full heart contraction cycle. The decision logic may be threshold based. The decision logic may use an analysis window covering at least one heart contraction cycle of, for instance 2 s and at least several tenths of a millimeter of depth range, for instance 1 mm. The variance of the velocity values may be larger than a signal threshold (ST). The signal threshold (ST) is a threshold on the variance values. It may be regarded as being a first threshold. When the variance values are larger than the first threshold, the likelihood that blood is between the device and the object is very high, hence there is no contact between the device and the object.

A fraction may further be determined as being the ratio between the number of variance of the velocity values larger than a first threshold and the total number of variance of the velocity values in the depth-time window. When the fraction with values between 0 and 1 has a large value, then the number of variance of the velocity values bigger than the first threshold is large and therefore the likelyhood of no contact between the device and the cardiac tissue is high.

Furthermore, a second threshold and a third threshold being larger than the second threshold may be defined. The significance of the second threshold is that when the fraction is smaller than the second threshold, then the likelihood that in the depth-time window the number of variance of velocity values larger than the first threshold is low, therefore it is certain that blood is not present between the device and the object. This leads to a continuous contact determination between the device and the object. When the fraction is larger than the third threshold, then the likelihood that in the depth-time window the number of variance of velocity values larger than the first threshold is high, therefore it is certain the presence of blood between the device and the object. In case the fraction is larger than the second threshold and smaller than the third threshold, it is likely that some blood is present between the device and the object, at least in some of the phases of the heart contraction, therefore the determined degree of contact is defined as an intermittent contact. This scheme can easily be extended to be able to output more contact levels instead of only three discrete indications. The signal percentage SP can be also used directly as an output of the contact assessment, in order to indicate the amount or degree of contact.

Preferably steps 81 and 82 are performed by the motion distribution determination unit, whereas steps 83 to 88 are performed by the contact determination apparatus. In an embodiment all the steps 81 to 88 can be performed by the system comprising the motion distribution determination unit and the contact determination apparatus.

Although in above described embodiments the tip of the catheter comprises four ultrasound transducers, in other embodiments the catheter tip can also comprise one, two, three or more than four ultrasound transducers. Moreover, although in above described embodiments the device is an ablation catheter, in other embodiments the device can also be another element like a needle.

In the above described embodiments the contact determination apparatus is adapted to determine a degree of contact between cardiac tissue and the device, in other embodiments the contact determination apparatus can also be adapted to detect a degree of contact between a device and another object not being cardiac tissue like tissue of another part, in particular, of another organ, of a living being like a person or an animal. The contact determination apparatus can also be used to determine a degree of contact between a device and a technical object.

Although in above described embodiments the degree of contact has been determined based on the provided motion distributions only, i.e. only on the motion in the surrounding of the device, in other embodiments in addition further information can be used for determining the degree of contact. For instance, the contact determination unit may be adapted to determine the degree of contact based on the provided motion distribution and further based on intensities of the received ultrasound signals. In particular, if the received ultrasound signals are A-mode lines, the A-mode lines can be averaged by using an average window of, for example 2 s, wherein the resulting average value may be compared with one or several thresholds for determining an intensity-based degree of contact. Also other intensity-based techniques can be used like the intensity-based technique disclosed in U.S. Pat. No. 5,840,030A. In addition, the degree of contact can be determined based on the provided motion distribution, in order to determine a motion-based degree of contact. These two determined degrees of contact can be combined, in order to determine a final degree of contact, by applying predefined combination rules. For instance, the predefined combination rules can define that the degree of contact is a continuous contact, only if both, the intensity-based degree of contact and the variance of the velocity based degree of contact, indicate a continuous contact.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Operations like the determination of motion distributions, the determination of the degree of contact, et cetera performed by one or several units or devices can be performed by any other number of units or devices. For example, steps 101 and 102 and/or steps 80 to 88 can be performed by a single unit or by any other number of different units. These operations and/or the control of the contact determination apparatus in accordance with the contact determination method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A contact determination apparatus for determining a degree of contact between a moving object, and a device that comprises an ultrasound transducer,
the contact determination apparatus adapted to be operatively coupled to the device through a motion distribution determination unit for providing ultrasound signals indicative of motion in a surrounding of the device,
wherein the contact determination apparatus is adapted to determine a variance of the velocity in the surrounding of the device comprising the moving object from the signals received from the motion distribution determination unit, and
wherein the contact determination apparatus configured to determine a degree of contact between the device and the moving object based on the variance of the velocity.

2. The contact determination apparatus according to claim 1, wherein the variance of the velocity values are determined for a depth-time window from signals indicative of motion in the surrounding of the device received from the motion distribution determination unit.

3. The contact determination apparatus according to claim 2, wherein the depth-time window is adapted to move in time.

4. The contact determination apparatus as defined in claim 2
wherein the contact determination apparatus is adapted to determine a fraction defined by the ratio between the number of variance of the velocity values being larger than a first threshold and the total number of variance of the velocity values in the depth-time window,
wherein the degree of contact is based on the determined fraction.

5. The contact determination apparatus as defined in claim 4, wherein the contact determination apparatus is adapted to determine the degree of contact by thresholding the determined fraction.

6. The contact determination apparatus as defined in claim 4, wherein the contact determination apparatus is adapted to provide a second threshold and a third threshold being larger than the second threshold, to determine that the degree of contact is a) a continuous contact, if the fraction is smaller than the second threshold, b) no contact, if the fraction is larger than the third threshold, and c) an intermittent contact otherwise.

7. The contact determination apparatus as defined in claim 1, wherein the contact determination apparatus is adapted to distinguish at least between a) a continuous contact between the device and the object, b) no contact between the device and the object, and c) an intermittent contact between the device and the object.

8. A system comprising the contact determination apparatus according to claim 1 and a motion distribution determination unit for providing signals indicative of motion in a surrounding of the device.

9. An energy application system comprising an energy source, an energy application device and the system according to claim 8,
wherein the energy application device is operatively coupled to the energy source,
wherein the energy application device further comprises an ultrasound transducer operatively coupled to the motion distribution determination unit,
wherein the contact determination apparatus is configured to determine the degree of contact between the energy application device and the moving object.

10. The energy application system according to claim 9, wherein the energy source is adapted to provide energy to the energy application device only when the contact determination apparatus determines a continuous contact between the energy application device) and the object.

11. The energy application system according to claim 10, further comprising a navigation unit.

12. A contact determination method for determining a degree of contact between a moving object, and a device that comprises an ultrasound transducer wherein the contact determination method comprises:
providing ultrasound from the device to a motion distribution determination unit, the ultrasound signals being indicative of motion in the surrounding of the device comprising an object,
determining a variance of the velocity from the motion distribution signals, and
determining a degree of contact between the device and the object based on the variance of the velocity.

13. An energy application method comprising:
navigating the energy application device to the object
determining the degree of contact between the energy application device and the object according to claim 12, and
performing energy application to the object when a continuous contact between the energy application device and the object is determined.

14. A computer-readable medium having stored a computer-executable program for determining a degree of contact between a device and a moving object, the computer program comprising program code means for causing a contact determination apparatus as defined in claim 1 to carry out the steps of the contact determination method when the computer program is run on a computer controlling the contact determination apparatus.
